(19) United States
(12) Patent Application Publication
(10) Pub. No.: US 2009/0176744 A1
(43) Pub. Date: Jul. 9, 2009

(54) Deuterated fingolimod

(75) Inventors: Julie F. Liu, Lexington, MA (US); Rose A. Persichetti, Stow, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: CoNCERT Pharmaceuticals, Inc., Lexington, MA (US)

(21) Appl. No.: 12/290,645

(22) Filed: Oct. 31, 2008

Related U.S. Application Data
(60) Provisional application No. 61/001,569, filed on Nov. 2, 2007.

(51) Int. Cl. A61K 31/661; C07C 211/27; A61K 31/138; C07F 9/06

(52) U.S. Cl. 514/114; 564/360; 514/653; 558/169

(57) ABSTRACT

This invention relates to novel compounds that are deuterated derivatives of fingolimod and pharmaceutically acceptable salts thereof. This invention also provides compositions comprising one or more compounds of this invention and a carrier and the use of the disclosed compounds and compositions in methods of treating diseases and conditions that are beneficially treated by administering a lysophospholipid edg1 (S1P1) receptor agonist, such as fingolimod.

### RELATED APPLICATION

This application claims the benefit of U.S. Provisional Application No. 61/001,569, filed on Nov. 2, 2007. The entire teachings of the above application is incorporated herein by reference.

Fingolimod, also known as 2-amino-2-[2-(4-octylphenyl)ethyl]-1,3-propanediol hydrochloride, a sphingosine-1-phosphate receptor agonist, acts as an immunomodulator by inducing lymphopenia through sequestration of circulating lymphocytes into secondary lymphoid tissues, thus preventing lymphocytes from moving into the transplanted or other affected tissues (Chiba, K et al., Transplant Proc., 2005, January-February, 37(1): 102-6).

Fingolimod is currently in phase III clinical trials for multiple sclerosis (MS).

In general, fingolimod has been found to be safe and well-tolerated (Kahan, B D et al., Transplantation, 2003, 76(7): 1079; Budde, K et al., Journal of the American Society of Nephrology, 2002, 13(14): 1073-1083; and Ferguson, R M et al., American Journal of Transplantation, 2003, 3(311): (Abs 624)). However, one clinical trial in which fingolimod was administered to renal transplant patients (Tedesco-Silva H et al., Transplantation, 2004, 77(12): 1826), showed a mild and transient reduction in heart rate associated with fingolimod treatment, reversible upon cessation of treatment.

Despite the beneficial activities of fingolimod, there is a continuing need for new compounds to treat the aforementioned diseases and conditions.

### SUMMARY OF THE INVENTION

This invention relates to novel compounds that are deuterated derivatives of fingolimod and pharmaceutically acceptable salts thereof. This invention also provides compositions comprising one or more compounds of this invention and a carrier and the use of the disclosed compounds and compositions in methods of treating diseases and conditions that are beneficially treated by administering a lysophospholipid edg1 (S1P1) receptor agonist, such as fingolimod.

### DETAILED DESCRIPTION OF THE INVENTION

The terms “ameliorate” and “treat” are used interchangeably and include both therapeutic treatment and prophylactic treatment (reducing the likelihood of development). Both terms mean decrease, suppress, attenuate, diminish, arrest, or stabilize the development or progression of a disease (e.g., a disease or disorder delineated herein), lessen the severity of the disease or improve the symptoms associated with the disease.

“Disease” means any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of fingolimod will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Wada, E et al., Seikagaku, 1994, 66: 15; Ganes, L Z et al., Comp Biochem Physiol Mol Integr Physiol, 1998, 119: 725.

In a compound of this invention, when a particular position is designated as having deuterium, it is understood that the abundance of deuterium at that position is substantially greater than the natural abundance of deuterium, which is 0.015%. Unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

In the compounds of the invention, any atom not specifically designated as a particular isotope is meant to represent any stable isotope of that atom unless otherwise stated. Unless otherwise stated, when a position is designated specifically as “H” or “hydrogen,” the position is understood to have hydrogen at its natural abundance isotopic composition.

The term “isotopic enrichment factor” as used herein means the ratio between the isotopic abundance and the natural abundance of that isotope.

In other embodiments, a compound of this invention has an isotopic enrichment factor for each deuterium present at a site designated as a potential site of deuteration on the compound of at least 3500 (52.5% deuterium incorporation), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The term “isotopologue” refers to a species that differs from a specific compound of this invention only in the isotopic composition thereof. Isotopologues can differ in the level of isotopic enrichment at one or more positions and/or in the positions(s) of isotopic enrichment.

The term “compound,” when referring to a compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

The structural formula depicted herein may or may not indicate whether atoms at certain positions are isotopically enriched. In a most general embodiment, when a structural formula is silent with respect to whether a particular position is isotopically enriched, it is to be understood that the stable isotopes at the particular position are present at natural abundance, or, alternatively, that that particular position is isotopically enriched with one or more naturally occurring stable isotopes. In a more specific embodiment, the stable isotopes are present at natural abundance at all positions in a compound not specifically designated as being isotopically enriched.

The invention also provides salts, solvates and hydrates of the compounds of the invention.

A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

As used herein, the term “hydrate” means a compound which further includes a stoichiometric or non-stoichiometric amount of water bound by non-covalent intermolecular forces.

As used herein, the term “solvate” means a compound which further includes a stoichiometric or non-stoichiometric amount of solvent such as water, acetone, ethanol, methanol, dichloromethane, 2-propanol, or the like, bound by non-covalent intermolecular forces.

The compounds of the present invention (e.g., compounds of Formula I), may contain an asymmetric carbon atom, for example, as the result of deuterium substitution or otherwise. As such, compounds of this invention can exist as either individual enantiomers, or mixtures of the two enantiomers. Accordingly, a compound of the present invention will include both racemic mixtures, and also individual respective stereoisomers that are substantially free from another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers, or less than “X”% of other stereoisomers (wherein X is a number between 0 and 100, inclusive) are present. Methods of obtaining or synthesizing an individual enantiomer for a given compound are well known in the art and may be applied as practicable to final compounds or to starting material or intermediates.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” refers to deuterium. “Stereoisomer” refers to both enantiomers and diastereomers.

Throughout this specification, a variable may be referred to generally (e.g., “each Y”) or may be referred to specifically (e.g., R¹, Y¹, Y², Y³, etc.). Unless otherwise indicated, when a variable is referred to generally, it is meant to include all specific embodiments of that particular variable.

### Therapeutic Compounds

The present invention provides a compound of Formula I:

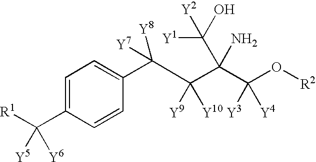

or a pharmaceutically acceptable salt thereof, wherein:

each Y is independently selected from H and D;

R¹ is —(CH₂)₆—CH₃, wherein from 1 to 15 hydrogen atoms are optionally replaced by deuterium atoms;

R² is selected from H and —P(O)(OH)₂; and

when each Y is H, R¹ contains at least one deuterium atom.

In one embodiment of a compound of Formula I, each methylene carbon of R¹ independently bears either 2 hydrogen or 2 deuterium atoms. Specific examples of R¹ include —(CH₂)₆—CD₃, —(CH₂)₅—CD₂CD₃, and —(CD₂)₆—CD₃.

Other embodiments of a compound of Formula I include those wherein:

a) each of Y¹, Y², Y³ and Y⁴ is the same;

b) each of Y⁵ and Y⁶ is the same;

c) each of Y⁷ and Y⁸ is the same;

d) each of Y⁹ and Y¹ is the same; and

e) R¹ is —(CH₂)₆—CD₃, wherein from 1 to 12 hydrogen atoms are optionally replaced by deuterium atoms.

Still other embodiments include a compound of Formula I having two or more of the properties set forth in a) through e), above. Such combinations include, but are not limited to: a) and b); a) and c); a) and d); b) and c); b) and d); d) and c); a), b) and c); a), b) and d); a), c) and d); b), c) and d); and a), b), c) and d).

In one specific embodiment, R² is —P(O)(OH)₂; and each of Y¹, Y², Y³ and Y⁴ is the same. In an even more specific embodiment, R² is —P(O)(OH)₂; each of Y¹, Y², Y³ and Y⁴ is the same; and the compound has one or more of the properties set forth in b) through e), above. For example, R² is —P(O)(OH)₂; each of Y¹, Y², Y³ and Y⁴ is the same in combination with one of the following: b); c); d); b) and c); b) and d); c) and d); and b), c) and d).

In another specific embodiment R¹ is selected from —(CH₂)₆—CD₃ and —(CD₂)₆—CD₃. In a more specific embodiment, R¹ is selected from —(CH₂)₆—CD₃ and —(CD₂)₆—CD₃, and the compound has one or more of the properties set forth in a) through d) above. For example, R¹ is selected from —(CH₂)₆—CD₃ and —(CD₂)₆—CD₃ in combination with one of the following: a); b); c); a) and b); a) and c); b) and c); a), b) and c); d); a) and d); b) and d); a), b) and d); d) and c); a), c) and d); b), c) and d); and a), b), c) and d).

In an even more specific embodiment, R¹ is selected from —(CH₂)₆—CD₃ and —(CD₂)₆—CD₃, R² is —P(O)(OH)₂; and each of Y¹, Y², Y³ and Y⁴ is the same. In yet another more specific embodiment, R¹ is selected from —(CH₂)₆—CD₃ and —(CD₂)₆—CD₃, R² is —P(O)(OH)₂; each of Y¹, Y², Y³ and Y⁴ is the same, and the compound has one or more of the properties set forth in b) through d), above.

In another embodiment R¹ is selected from —(CH₂)₆—CD₃ and —(CD₂)₆—CD₃; and R² is hydrogen. In one aspect of this embodiment, each of Y¹, Y², Y³ and Y⁴ is the same, each of Y⁵ and Y⁶ is the same, each of Y⁷ and Y⁸ is the same, and each of Y⁹ and Y¹⁰ is the same. In another aspect of this embodiment, each Y is deuterium. In still another aspect of this embodiment, each Y is hydrogen.

In another set of embodiments, in any of the embodiments set forth above, the compound has the (S) configuration at the carbon bearing the NH₂ group.

In a specific embodiment, the compound is selected from:

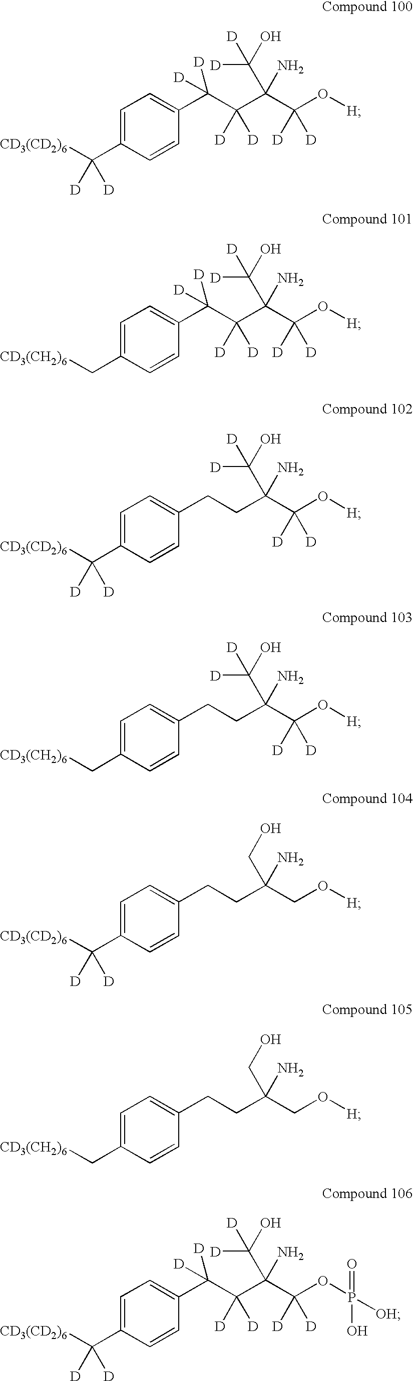

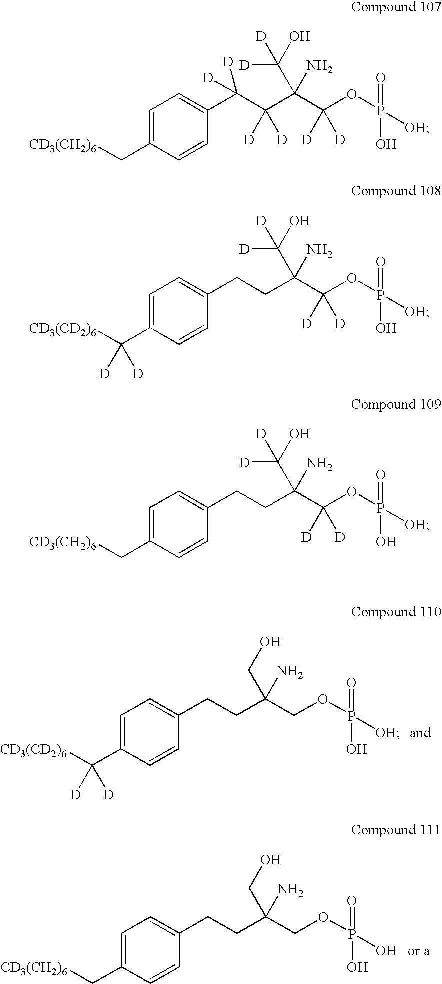

pharmaceutically acceptable salt of any of the foregoing.

In another specific embodiment, the compound is:

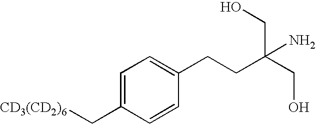

Compound 112 or a pharmaceutically acceptable salt thereof.

In another set of embodiments, any atom not designated as deuterium in any of the embodiments set forth above is present at its natural isotopic abundance.

The synthesis of compounds of Formula I can be readily achieved by synthetic chemists of ordinary skill. Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure. Relevant procedures and intermediates are disclosed, for instance in Chiba, K et al., Drugs Fut 1997, 22(1): 18; Adachi, K et al., Bioorg Med Chem Lett, 1995, 5(8): 853; Durand, P et al., Synthesis, (Stuttgart) 2000, 4: 505; Kalita, B et al., Syn Lett, (Stuttgart) 2001, 9: 1411; Kim, S et al., Synthesis, 2006, 5: 753-755; Lu, X et al., Tetrahedron Letters, 2006, 47(5): 825-827; Takeda, S et al., Tetrahedron Letter's 2005, 46(31): 5169-5172; Albert, R et al., J Med Chem, 2005, 48(16): 5373-5377; Kiuchi, M et al., Bioorg Medi Chem, 2005, 13(2): 425-432; Hale, J et al., Bioorg Med Chem, 2004, 12(18): 4803-4807; and in PCT patent publications WO 1994008943 and WO 2000053569. The schemes below illustrate how the present compounds may be prepared.

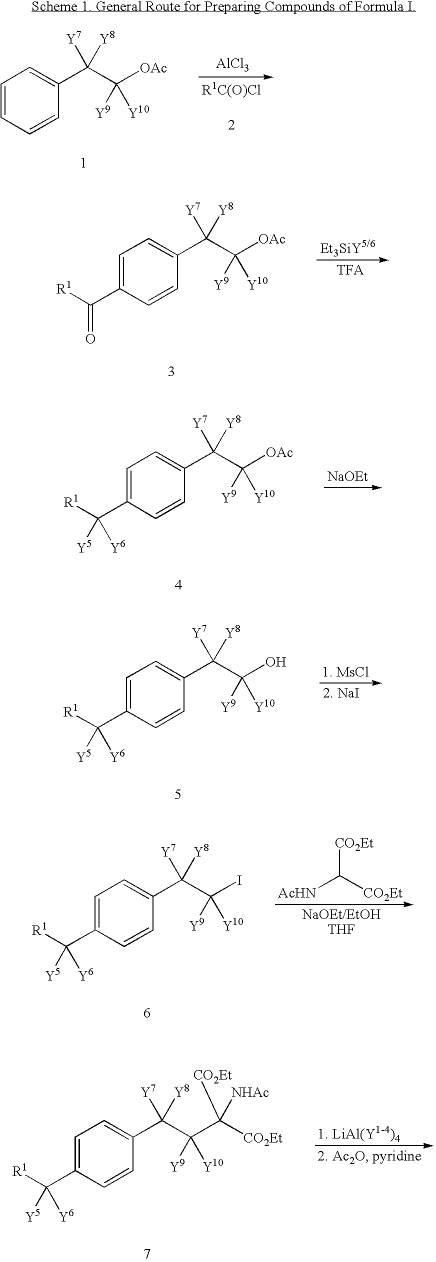

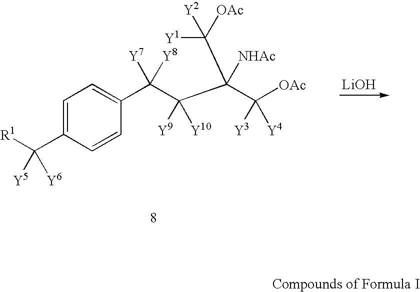

Scheme 1 shows a general route to preparing compounds of Formula I. As described generally in the fingolimod literature cited above, appropriately-deuterated acetate 1 undergoes Friedel-Crafts acylation with appropriately-deuterated acyl chloride 2 in the presence of AlCl₃ to afford ketone 3. Ketone 3 is reduced with either triethylsilane or commercially-available triethyl(silane-d) to provide acetate 4. Hydrolysis of the acetate yields alcohol 5, which is converted to the mesylate with methanesulfonyl chloride and displaced with sodium iodide to afford iodide 6. Reaction of iodide 6 with commercially-available diethyl acetamidomalonate in the presence of sodium ethoxide yields ester 7. Reduction with either LiAlH₄ or LiAlD₄, followed by acylation with acetic anhydride provides acetate 8. Hydrolysis of the acetate groups with lithium hydroxide provides compounds of Formula I wherein R² is hydrogen, Y⁵ and Y⁶ are the same; and each of Y¹, Y², Y³, and Y⁴ is the same.

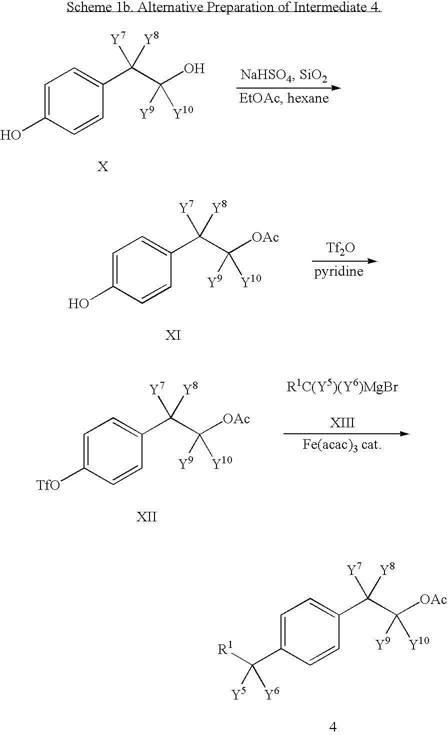

Scheme 1b depicts an alternate synthesis of intermediate 4, which can be further converted to compounds of Formula I following the route shown in Scheme 1. This alternate synthesis follows the general methods of Seidel, G.; et al. JOC, 2004, 69(11), 3950-3952. Appropriately-deuterated alcohol X is acetylated to afford XI. Treatment with triflic anhydride provides triflate XII. Iron-catalyzed coupling of appropriately-deuterated Grignard reagent XIII yields intermediate 4.

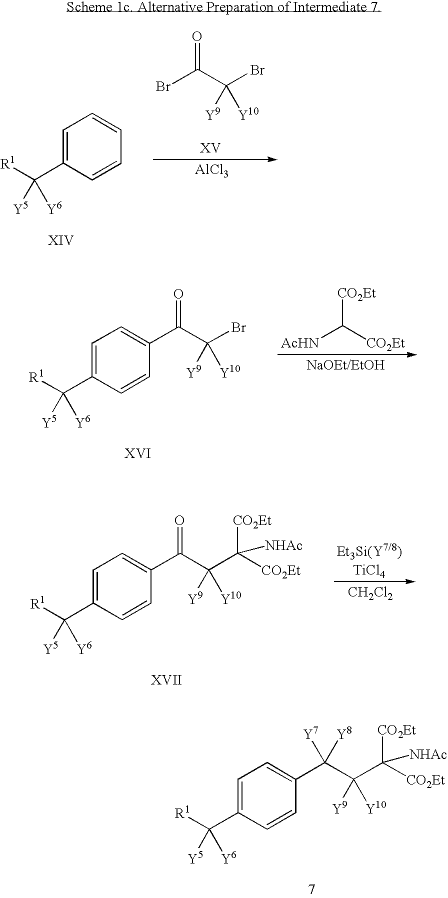

Scheme 1c depicts an alternate synthesis of intermediate 7, which can be further converted to compounds of Formula I following the route shown in Scheme 1. This alternate synthesis follows the general methods of Durand, P; et al. Synthesis 2000, 4, 505-506, and later modifications by Foss, F W; et al. BMCL 2005, 15, 4470-4474. Appropriately-deuterated XIV is acylated with appropriately-deuterated XV to afford XVI. Reaction of XVI with commercially-available diethyl acetamidomalonate in the presence of sodium ethoxide yields XVII. Treatment with either triethylsilane or commercially-available triethyl(silane-d) affords intermediate 7.

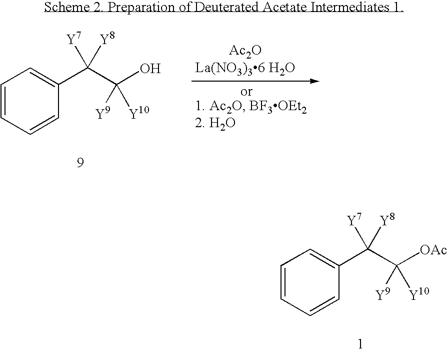

Deuterated acetates 1 for use in Scheme 1 can be synthesized as set forth in Scheme 2. Following the general methods found in Reddy, T S et al., Tet Lett, 2006, 47(38): 6825-6829, deuterated alcohol 9 is acylated with acetic anhydride in the presence of La(NO₃)₃·6H₂O to afford intermediate 1. Alternatively, following the methods of Martinez-Pascual, R et al., Synth Comm, 2004, 34(24): 4591-4596, alcohol 9 is treated with acetic anhydride and BF₃·OEt₂ followed by water to afford intermediate 1. One example of a commercially-available alcohol to be used as alcohol 9 is 2-phenylethan-1,1,2,2-d₄-ol (PhCD₂CD₂OH). This alcohol is used to produce a compound of Formula I wherein Y⁷, Y⁸, Y⁹ and Y¹⁰ are simultaneously deuterium.

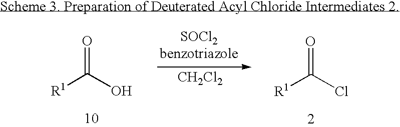

Deuterated acyl chlorides 2 for use in Scheme 1 can be synthesized as set forth in Scheme 3. Following the method found in Chaudhari, S S et al., Syn Lett, 1999, 11: 1763-1765, deuterated carboxylic acids 10 are treated with a 1:1 mixture of thionyl chloride and benzotriazole in CH₂Cl₂ to afford acyl chlorides 2. One example of a commercially available deuterated carboxylic acid is octanoic-d₁₅ acid (CD₃(CD₂)₆COOH), which may be used as carboxylic acid 10 in Scheme 3 to ultimately produce compounds of Formula I wherein R¹ is CD₃(CD₂)₆. Another example is commercially-available octanoic-8,8,8-d₃ acid (CD₃(CH₂)₆COOH), which may be used as carboxylic acid 10 in Scheme 3 to ultimately produce compounds of Formula I wherein R¹ is CD₃(CH₂)₆.

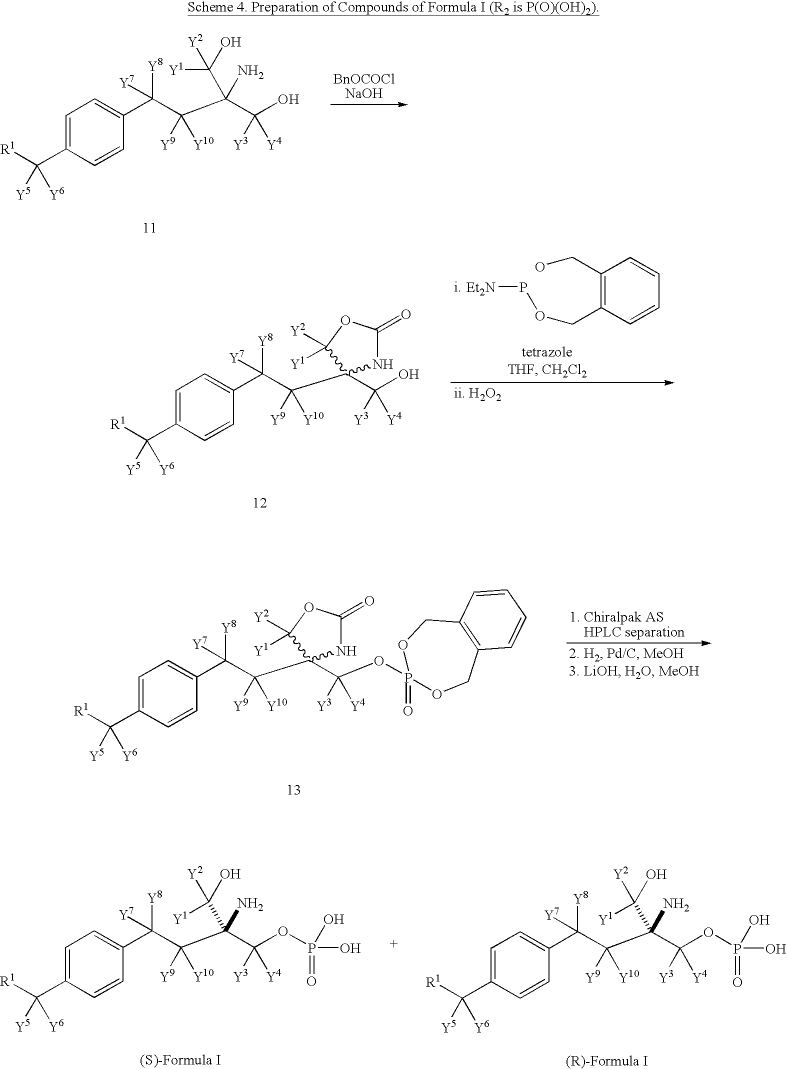

Scheme 4 shows a general synthetic route to compounds of Formula I, wherein R² is P(O)(OH)₂. As described generally in Albert, R et al., J Med Chem, 2005, 48: 5373-5377, deuterated compounds of Formula I, wherein Y¹, Y², Y³ and Y⁴ are the same (11), is treated with benzylchloroformate and sodium hydroxide to afford racemic oxazolidinone 12. Phosphorylation of the remaining hydroxyl group with commercially-available o-xylylene N,N-diethylphosphoramidite, followed by oxidation with hydrogen peroxide, yields racemic protected phosphate 13. Separation of the R and S enantiomers via chiral HPLC, then cleavage of the phosphate protecting group of each enantiomer with H₂ and Pd/C, followed by hydrolysis of the oxazolidinone with LiOH provides the individual enantiomeric compounds of Formula I wherein R² is P(O)(OH)₂.

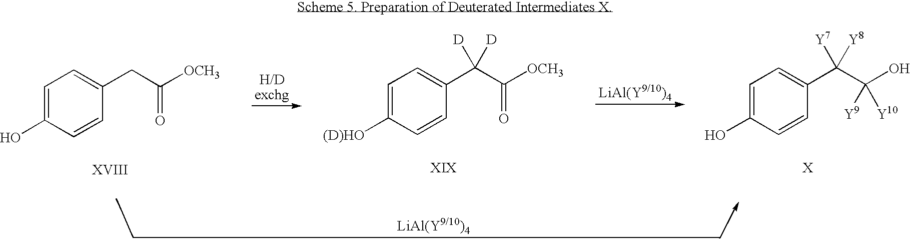

Scheme 5 depicts the preparation of deuterated intermediates X for Scheme 1b. If desired, hydrogen/deuterium exchange of commercially-available methyl 4-hydroxyphenylacetate XVIII is performed either with NaOMe/MeOD, or with triazabicyclo[4.4.0]dec-5-ene “TBD” and CDCl₃ according to the methods of Sabot, C et al., JOC, 2007, 72(13): 5001-5004, to provide ester XIX. Reduction of XIX with LiAlH₄ or LiAlD₄ affords X wherein Y⁷ and Y⁸ are deuterium. Alternatively, methyl 4-hydroxyphenylacetate XVIII is reduced directly with LiAlH₄ or LiAlD₄ to afford X wherein Y⁷ and Y⁸ are hydrogen.

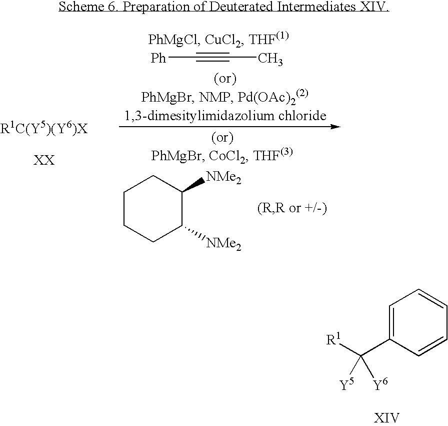

Scheme 6 depicts three methods for converting appropriately-deuterated XX (wherein X is Cl, Br, or I) to deuterated intermediates XIV (cf. Scheme 1c). These three approaches follow the general literature methods of:
(1) Terao, J et al., Angewandte Chemie, International Edition, 2007, 46(12): 2086-2089.
(2) Frisch, A. C et al., Journal of Organometallic Chemistry, 2003, 687(2): 403-409.
(3) Ohmiya, H et al., J. Am. Chem. Soc., 2006, 128(6): 1886-1889.
   Examples of useful deuterated halides XX include, but are not limited to, commercially-available CD₃(CD₂)₆CD₂Br; CD₃(CD₂)₆CD₂1; and CD₃(CH₂)₆CH₂Br. Another useful compound XX is CD₃(CD₂)₆CH₂Br, which may be synthesized from commercially-available CD₃(CD₂)₆COOH using LiAlH₄ and HBr according to the general methods of Boden, N et al., JCS Perkins Trans 1, 1983, 3: 543-551.

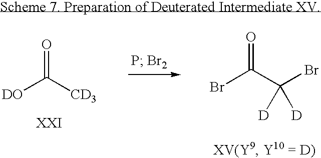

Scheme 7 depicts the preparation of a useful deuterated version of intermediate XV (cf. Scheme 1c) wherein Y⁹ and Y¹⁰ are both deuterium. Commercially-available acetic acid-d4 is treated with red phosphorus and bromine according to the procedure of Goerger, M M et al., J. Org. Chem., 1988, 53(14): 3148-53 to provide XV wherein Y⁹ and Y¹⁰ are both deuterium.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art.

Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene T W et al., Protective Groups in Organic Synthesis, 3^{rd} Ed., John Wiley and Sons (1999); Fieser L et al., Fieser and Fieser's Reagents for Organic Synthesis, John Wiley and Sons (1994); and Paquette L, ed., Encyclopedia of Reagents for Organic Synthesis, John Wiley and Sons (1995) and subsequent editions thereof.

Combinations of substituents and variables envisioned by this invention are only those that result in the formation of stable compounds.

### Compositions

The invention also provides pyrogen-free compositions comprising an effective amount of a compound of Formula I (e.g., including any of the formulae herein), or a pharmaceutically acceptable salt of said compound; and an acceptable carrier. Preferably, a composition of this invention is formulated for pharmaceutical use (“a pharmaceutical composition”), wherein the carrier is a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide: See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

In another embodiment, a composition of this invention further comprises a second therapeutic agent. The second therapeutic agent may be selected from any compound or therapeutic agent known to have or that demonstrates advantageous properties when administered with a compound having the same mechanism of action as fingolimod. Such agents include those indicated as being useful in combination with fingolimod, including but not limited to, those described in WO 1994008943, WO 2003097028, WO 2005105146, and WO 2007041368.

Preferably, the second therapeutic agent is an agent useful in the treatment or prevention of a disease or condition selected from rejection after organ or bone marrow transplantation, multiple sclerosis, inflammatory bowel disease, cancer, ulcerative colitis or another disease requiring immunosuppression.

In one embodiment, the second therapeutic agent is selected from tacrolimus, a corticosteroid, and a cyclosporin.

In another embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to treat (therapeutically or prophylactically) the target disorder. For example, and effective amount is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., (1966) Cancer Chemother. Rep 50: 219. Body surface area may be approximately determined from height and weight of the patient. See, e.g., Scientific Tables, Geigy Pharmaceuticals, Ardsley, N.Y., 1970, 537.

In one embodiment, an effective amount of a compound of this invention can range from about 1.25 μg to about 50 mg per treatment. In more specific embodiments the range is from about 12.5 μg to 25 mg, or from 25 μg to 10 mg, or most specifically from about 0.125 mg to 5 mg per treatment. Treatment typically is administered once daily.

Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician. For example, guidance for selecting an effective dose can be determined by reference to the prescribing information for fingolimod.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this invention. When this occurs, it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

In another embodiment, the invention provides a method of modulating the activity of the S1P1 receptor in a cell, or specifically in a lymphocyte or endothelial cell, comprising contacting such cell with one or more compounds of Formula I herein.

According to another embodiment, the invention provides a method of treating a disease that is beneficially treated by fingolimod comprising the step of administering to a patient in need thereof an effective amount of a compound or a composition of this invention. Such diseases are well known in the art and are disclosed in, but not limited to the following patents and published applications: WO 1994008943, WO 2001001978, WO 2003009836, WO 2003035068, WO 2003097028, WO 2004010987, WO 2004028521, WO 2004110421, WO 2005002559, WO 2005025553, WO 2005058295, WO 2005105146, WO 2006010630, WO 2006072562, WO 2006094705, and WO 2006102611. Such diseases include, but are not limited to, rejection after organ or bone marrow transplantation (e.g., anti-rejection therapy), immunosuppressive sustention therapy, eye diseases such as Behcet's disease and uveitis, and dermatitis inclusive of psoriasis, atopic dermatitis, contact dermatitis and allergic dermatitis; resistance or rejection in organ or tissue transplantation (e.g., transplantation of heart, kidney, liver, lung, bone marrow, cornea, pancreas, small intestine, limb, muscle, nerves, fatty marrow, duodenum, skin and pancreatic islet cell, and xeno-transplantation), graft-versus-host (GvH) diseases by bone marrow or small intestine transplantation, autoimmune diseases such as rheumatoid arthritis, systemic lupus erythematosus, nephrotic syndrome lupus, Hashimoto's thyroiditis, multiple sclerosis, myasthenia gravis, type I diabetes mellitus, type II adult onset diabetes mellitus, uveitis, nephrotic syndrome, steroid-dependent and steroid-resistant nephrosis, palmoplantar pustulosis, allergic encephalomyelitis, glomerulonephritis, etc., and infectious diseases caused by pathogenic microorganisms; inflammatory, proliferative and hyperproliferative skin diseases and cutaneous manifestations of immunologically-mediated illnesses such as psoriasis, psoriatic arthritis, atopic eczema (atopic dermatitis), contact dermatitis and further eczematous dermatitises, seborrheic dermatitis, lichen planus, pemphigus, bullous pemphigoid, epidermolysis bullosa, urticaria, angioedemas, vasculitides, erythemas, cutaneous eosinophils, acne, alopecia greata, eosinophilic fascitis, and atherosclerosis, hair revitalizing, such as in female or male pattern alopecia, or senile alopecia; respiratory diseases, for example, sarcoidosis, fibroid lung, idiopathic interstitial pneumonia, and reversible obstructive airways disease, including conditions such as asthma, including bronchial asthma, infantile asthma, allergic asthma, intrinsic asthma, extrinsic asthma and dust asthma, particularly chronic or inveterate asthma (e.g., late asthma and airway hyperresponsiveness), bronchitis and the like, treating hepatic injury associated with ischemia, certain eye diseases such as conjunctivitis, keratoconjunctivitis, keratitis, vernal conjunctivitis, uveitis associated with Behcet's disease, herpetic keratitis, conical cornea, dystrophia epithelialis cornea, keratoleukoma, ocular pemphigus, Mooren's ulcer, scleritis, Graves' ophthalmopathy, severe intraocular inflammation and the like, inflammation of mucosa or blood vessels (e.g., leukotriene B4-mediated diseases, gastric ulcers, vascular damage caused by ischemic diseases and thrombosis, ischemic bowel disease, inflammatory bowel disease (e.g., Crohn's disease and ulcerative colitis), necrotizing enterocolitis), or intestinal lesions associated with thermal burns, renal diseases including interstitial nephritis, Goodpasture's syndrome, hemolytic uremic syndrome and diabetic nephropathy; nervous diseases selected from multiple myositis, Guillain-Barre syndrome, Meniere's disease and radiculopathy; endocrine diseases including hyperthyroidism and Basedow's disease; hematic diseases including pure red cell aplasia, aplastic anemia, hypoplastic anemia, idiopathic thrombocytopenic purpura, autoimmune hemolytic anemia, agranulocytosis and anerythroplasia; bone diseases including osteoporosis; respiratory diseases including sarcoidosis, fibroid lung and idiopathic interstitial pneumonia; skin diseases including dermatomyositis, vitiligo vulgaris, ichthyosis vulgaris, photoallergy sensitivity and cutaneous T cell lymphoma; circulatory diseases including arteriosclerosis, aortitis, polyarteritis nodosa and myocardosis; collagen disease including scleroderma, Wegener's granuloma and Sjogren's syndrome; adiposis; eosinophilic fascitis; periodontal disease; nephrotic syndrome; hemolytic uremic syndrome: and muscular dystrophy, diseases including intestinal inflammations or allergies such as Coeliac disease, proctitis, eosinophilic gastroenteritis, mastocytosis, Crohn's disease or ulcerative colitis; and food related allergic diseases which have symptomatic manifestation remote from the gastrointestinal tract, for example, migraine, rhinitis and eczema; heart diseases, including chronic heart failure, congestive heart failure, complications of cardiovascular surgery, peri-operative hypertension, unstable angina, and acute myocardial infarction; viral myocarditis and viral diseases induced by viral myocarditis; dementia or brain degeneration, beta-amyloid-related inflammatory diseases or disorders such as, Alzheimer disease, amyloidosis, Lewy Body diseases, multi-infarct dementia, Pick's disease or cerebral atherosclerosis; vascular permeability disorders and undesirable vascular endothelial cell apoptosis, as well as for the promotion of angiogenesis including endothelial injury, thrombocytopenia, atherosclerosis, ischemic cardiovascular disease and ischemic peripheral vascular diseases or disorders, for example those associated with diabetes, Dengue hemorrhagic fever, acute respiratory distress syndrome, vascular leak syndrome, sepsis or autoimmune vasculitis; pain; solid tumors, for example tumor invasiveness, and particularly inhibiting or controlling deregulated angiogenesis; ophthalmic disorders, notably those characterized by apoptosis-induced retinal/corneal cell degeneration, including ischemic retinopathies such as anterior ischemic optic neuropathy, optic neuritis, wet and dry age-related macular degeneration, diabetic retinopathy, diabetic and cystoid macular edema, proliferative diabetic retinopathy and retinal detachment; regulating neurogenesis (non-embryonic neural stem and progenitor cell activity) in the treatment of nervous system injury or disease including Parkinson's disease, Huntington's disease, Alzheimer's disease, amyotrophic lateral sclerosis, spinal ischemia, ischemic stroke, spinal cord injury, cancer-associated brain or spinal cord injury, Shy-Drager syndrome and progressive supranuclear palsy; fungal infection; BCR/ABL-mediated leukemia, including chronic myelogenous leukemia (CML), particularly the blast crisis stage of CML, Philadelphia-positive acute lymphoblastic leukemia (Ph′-ALL), and refractory leukemias; and treatment of hepatitis C or chronic hepatitis C(HCV).

In one particular embodiment, the method of this invention is used to treat a disease or condition selected from multiple sclerosis (MS), inflammatory bowel disease, cancer, and ulcerative colitis, or to prevent rejection following kidney transplantation in a patient in need thereof.

Identifying a patient in need of such treatment can be in the judgment of a patient or a health care professional and can be subjective (e.g. opinion) or objective (e.g. measurable by a test or diagnostic method).

In another embodiment, any of the above methods of treatment comprises the further step of co-administering to the patient one or more second therapeutic agents. The choice of second therapeutic agent may be made from any second therapeutic agent known to be useful for co-administration with fingolimod. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

In particular, the combination therapies of this invention include a method of preventing rejection following renal transplantation comprising the step of co-administering to a patient in need thereof a pharmaceutical composition comprising a compound of Formula I; and a pharmaceutical composition comprising a second therapeutic agent selected from tacrolimus, corticosteroids, and cyclosporins.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a patient does not preclude the separate administration of that same therapeutic agent, any other second therapeutic agent or any compound of this invention to said patient at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula I alone or together with one or more of the above-described second therapeutic agents in the manufacture of a medicament, either as a single composition or as separate dosage forms, for treatment or prevention in a patient of a disease, disorder or symptom set forth above. Another aspect of the invention is a compound of Formula I for use in the treatment or prevention in a patient of a disease, disorder or symptom thereof delineated herein.

### Pharmaceutical Kits

The present invention also provides kits for use to treat multiple sclerosis (MS), inflammatory bowel disease, cancer, or ulcerative colitis, or to prevent rejection following kidney transplantation. These kits comprise (a) a pharmaceutical composition comprising a compound of Formula I or a salt thereof, wherein said pharmaceutical composition is in a container; and (b) instructions describing a method of using the pharmaceutical composition to treat multiple sclerosis (MS), inflammatory bowel disease, cancer, and ulcerative colitis, or to prevent rejection following kidney transplantation.

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. In one embodiment, the container is a blister pack.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

In certain embodiment, the kits of this invention may comprise in a separate vessel of container a pharmaceutical composition comprising a second therapeutic agent, such as one of those listed above for use for co-administration with a compound of this invention.

### Evaluation of Metabolic Stability

Certain in vitro liver metabolism studies have been described previously in the following references, each of which is incorporated herein in their entirety: Obach, R S, Drug Metab Disp, 1999, 27:1350; Houston, J B et al., Drug Metab Rev, 1997, 29:891; Houston, J B, Biochem Pharmacol, 1994, 47:1469; Iwatsubo, T et al., Pharmacol Ther, 1997, 73:147; and Lave, T, et al., Pharm Res, 1997, 14:152.

Microsomal Assay: The metabolic stability of compounds of Formula I is tested using pooled liver microsomal incubations. Full scan LC-MS analysis is then performed to detect major metabolites. Samples of the test compounds, exposed to pooled human liver microsomes, are analyzed using HPLC-MS (or MS/MS) detection. For determining metabolic stability, multiple reaction monitoring (MRM) is used to measure the disappearance of the test compounds. For metabolite detection, Q1 full scans are used as survey scans to detect the major metabolites.

Human liver microsomes (20 mg/mL) are obtained from Xenotech, LLC (Lenexa, Kans.). β-nicotinamide adenine dinucleotide phosphate, reduced form (NADPH), magnesium chloride (MgCl₂), and dimethyl sulfoxide (DMSO) are purchased from Sigma-Aldrich. The incubation mixtures are prepared according to the Table:

TABLE
Reaction Mixture Composition for
Human Liver Microsome Study

| | | |
|---|---|---|
| Liver Microsomes | 3.0 | mg/mL |
| Potassium Phosphate, pH 7.4 | 100 | mM |
| Magnesium Chloride | 10 | mM |

Determination of Metabolic Stability: Two aliquots of this reaction mixture are used for a compound of this invention. The aliquots are incubated in a shaking water bath at 37° C. for 3 minutes. The test compound is then added into each aliquot at a final concentration of 0.5 μM. The reaction is initiated by the addition of cofactor (NADPH) into one aliquot (the other aliquot lacking NADPH serves as the negative control). Both aliquots are then incubated in a shaking water bath at 37° C. Fifty microliters (50 μL) of the incubation mixtures are withdrawn in triplicate from each aliquot at 0, 5, 10, 20, and 30 minutes and combined with 50 μL of ice-cold acetonitrile to terminate the reaction. The same procedure is followed for fingolimod and an appropriate positive control. Testing is done in triplicate.

Data analysis: The in vitro half-lives (t_{1/2}s) for test compounds are calculated from the slopes of the linear regression of % parent remaining (In) vs incubation time relationship.

in vitro t_{1/2}=0.693/k

k=−[slope of linear regression of % parent remaining(ln) vs incubation time]

Data analysis is performed using Microsoft Excel Software.

SUPERSOMES™ Assay. Various human cytochrome P450-specific SUPERSOMES™ are purchased from Gentest (Woburn, Mass., USA). A 1.0 mL reaction mixture containing 25 pmole of SUPERSOMES™, 2.0 mM NADPH, 3.0 mM MgCl, and 1 μM of a test compound in 100 mM potassium phosphate buffer (pH 7.4) was incubated at 37° C. in triplicate. Positive controls contain 1 μM of fingolimod instead of a test compound. Negative controls used Control Insect Cell Cytosol (insect cell microsomes that lacked any human metabolic enzyme) purchased from GenTest (Woburn, Mass., USA). Aliquots (50 μL) are removed from each sample and placed in wells of a multi-well plate at various time points (e.g., 0, 2, 5, 7, 12, 20, and 30 minutes) and to each aliquot is added 50 μL of ice cold acetonitrile with 3 μM haloperidol as an internal standard to stop the reaction.

Plates containing the removed aliquots are placed in −20° C. freezer for 15 minutes to cool. After cooling, 100 μL of deionized water is added to all wells in the plate. Plates are then spun in the centrifuge for 10 minutes at 3000 rpm. A portion of the supernatant (100 μL) is then removed, placed in a new plate and analyzed using Mass Spectrometry.

### EXAMPLES

### Example 1

### Synthesis of d₁₅-Octanoyl Chloride (21)

Intermediate 21 was prepared as outlined in Scheme 8 below. Details of the synthesis follow.

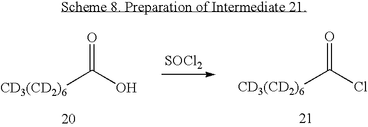

Synthesis of (Octanoyl-d₁₅) chloride (21). A mixture of octanoic-d₁₅-acid (4.00 g, 25.2 mmol, 1 equiv, CDN, 98.7 atom % D) and thionyl chloride (2.1 mL, 28.7 mmol, 1.4 equiv) were heated to reflux, neat, for 2 hours (h). The reaction mixture was cooled to room temperature (rt), and excess thionyl chloride was removed under reduced pressure to give crude 21 which was used without purification.

### Example 2

### Synthesis of 2-amino-2-(4-(2,2,3,3,4,4,5,5,5,6,6,7,7,8,8,8-d₁₅-octyl)phenethyl)propane-1,3-diol (112)

Compound 112 was prepared as outlined in Scheme 9 below. Details of the synthesis are set forth below.

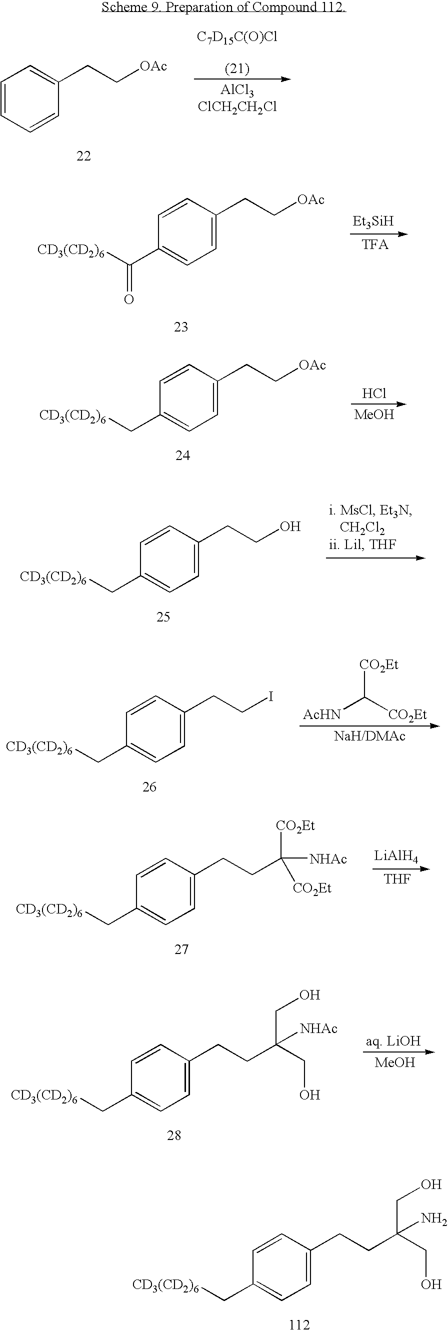

Synthesis of 4-(Octanoyl-d₁₅)phenethyl acetate (23). A suspension of aluminum chloride (5.35 g, 40.1 mmol, 1.6 equiv) in 1,2-dichloroethane (75 mL) was cooled to 0° C. in an ice-water bath. Commercially-available phenethyl acetate 22 (4.0 mL, 25.2 mmol, 1 equiv) and crude 21 (25.2 mmol, 1 equiv) were added together dropwise as a solution in 1,2-dichloroethane (30 mL) over 10 minutes (min) during which time the color became dark red/brown. The reaction was allowed to warn to rt and was stirred overnight. The reaction was quenched by the addition of D₂O (100 mL, Cambridge Isotope Labs, 99 atom % D). The resulting mixture was transferred to a separatory funnel, extracted with MTBE (2×, 350 mL total), and the organic layers were combined. The organic solution was washed with brine (300 mL), dried over Na₂SO₄, filtered, and evaporated under reduced pressure to give a brown oil. The crude reaction product was purified using an Analogix automated chromatography system eluting with a gradient of 10% EtOAc/heptanes to 50% EtOAc/heptanes over 45 min. Fractions containing product were concentrated under reduced pressure to give 5.12 g (66%) of 23 as a clear, colorless oil.

Synthesis of 4-(2,2,3,3,4,4,5,5,6,6,7,7,8,8,8-d₁₅-Octyl)phenethyl acetate (24). To a solution of 23 (3.0 g, 9.8 mmol, 1 equiv) in TFA (60 mL) was added triethylsilane (3.2 mL, 19.7 mmol, 2 equiv) dropwise via syringe over 10 min. The reaction was stirred at rt for 2 h, then was concentrated under reduced pressure to remove most of the volatile materials. The residue was dissolved in EtOAc (100 mL) and the resulting solution washed with 1 N NaOH (100 mL) followed by brine (100 mL). The organic layer was dried over Na₂SO₄, filtered, and concentrated under reduced pressure to give 2.86 g (100%) of crude 24 as a clear, slightly yellow oil. Crude 24 was used without further purification.

Synthesis of 4-(2,2,3,3,4,4,5,5,6,6,7,7,8,8,8-d₁₅-Octyl)phenyl ethanol (25). To a solution of crude 24 (2.50 g, 8.6 mmol, 1 equiv) in MeOH (200 mL) was added over 10 min a solution of 2N HCl in ether (50 mL, 100 mmol, 11.6 equiv). The reaction mixture was stirred at rt for 3 h, then was concentrated under reduced pressure to a volume of approximately 10 mL. The residue was diluted with EtOAc (100 mL) and the resulting solution was washed with satd. aq. NaHCO₃ (2×, 200 mL total), then brine (100 mL). The organic layer was dried over Na₂SO₄, filtered, and concentrated under reduced pressure to give 2.10 g (98%) of crude 25 as a clear, slightly yellow oil. Crude 25 was used without further purification.

Synthesis of 1-(2-Iodoethyl)-4-(2,2,3,3,4,4,5,5,6,6,7,7,8,8,8-d₁₅-octyl)benzene (26). A solution of methanesulfonyl chloride (1.03 g, 8.2 mmol, 1.2 equiv) in CH₂Cl₂ (10 mL) was added dropwise over 15 min to a solution of crude 25 (1.70 g, 6.8 mmol, 1 equiv) and triethylamine (1.17 g, 10.9 mmol, 1.6 equiv) in CH₂Cl₂ (40 mL). During the addition, the reaction temperature increased from 25° C. to 31° C. and the yellow color intensified. The reaction mixture was stirred for 1.5 h at rt, then was quenched by the addition of saturated aqueous (satd. aq.) NaHCO₃ (50 mL). The biphasic mixture was transferred to a separatory funnel and the phases were separated. The organic phase was dried over Na₂SO₄, filtered, and concentrated under reduced pressure to a yellow/brown oil. The crude oil was dissolved in THF (50 mL) and LiI (2.73 g, 20.4 mmol, 3 equiv) was added. The reaction mixture was stirred for 4 h in the absence of light, then was concentrated under reduced pressure. The resulting white solid was suspended in pentane (100 mL) and stirred vigorously for 30 min. The mixture was filtered through a small pad of silica gel topped with a small pad of Celite, and the pad was washed with hexanes (100 mL). The filtrate was concentrated under reduced pressure to give 1.93 g (79%) of crude 26 as a clear colorless oil. Crude 26 was used without further purification.

Synthesis of Diethyl 2-acetamido-2-(4-(2,2,3,3,4,4,5,5,6,6,7,7,8,8,8-d₁₅-octyl)phenethyl)malonate (27). To a solution of diethyl acetamidomalonate (2.85 g, 13.3 mmol, 6 equiv) in N,N-dimethylacetamide “DMAc” (38 mL) was added 60% sodium hydride (0.53 g, 13.3 mmol, 6 equiv) in several portions. The reaction was stirred for 30 min until the evolution of H₂ had ceased. A small increase in reaction temperature was observed during the stirring. 26 (0.75 g, 2.2 mmol, 1 equiv) was added and the reaction heated at 90° C. for 18 h. The reaction was cooled to rt, quenched with H₂O (50 mL), and extracted with MTBE (4×, 300 mL total). The combined organics were washed with brine (250 mL), dried over Na₂SO₄, filtered, and concentrated under reduced pressure to a yellow oil. The crude product was purified using an Analogix automated chromatography system eluting with 10% EtOAc/heptanes for 8 min followed by a gradient of 10% EtOAc/heptanes to 60% EtOAc/heptanes over 40 min. Fractions containing the product were concentrated under reduced pressure to give 550 mg (56%) of 27 as a clear, colorless oil.

Synthesis of N-(1-Hydroxy-2-(hydroxymethyl)-4-(4-(2,2,3,3,4,4,5,5,6,6,7,7,8,8,8-d₁₅-octyl)phenyl)butan-2-yl)acetamide (28). A solution of 1M LiAlH₄ in THF (3.3 mL, 3.3 mmol, 3 equiv) was diluted with additional THF (8 mL), then was cooled to 0° C. To the LiAlH₄ solution was added a solution of 27 (0.50 g, 1.1 mmol, 1 equiv) in THF (4 mL) dropwise via syringe over 15 min. The reaction mixture was allowed to warm to rt, then was stirred for 2 h. The resulting mixture was cooled to 0° C. and quenched by the addition of satd. aq. Na₂SO₄ (10 mL). The resulting turbid solution was filtered through a pad of Celite, the pad was washed with MeOH (100 mL), and the filtrate was concentrated under reduced pressure to a volume of approximately 10 mL. The residual aqueous solution was extracted with EtOAc (3×, 150 mL total). The combined organics were washed with brine (150 mL), dried over Na₂SO₄, filtered, and concentrated under reduced pressure to yield an off-white solid. The crude reaction product was purified via silica gel chromatography eluting first with CH₂Cl₂, then with a gradient of 0-5% MeOH in CH₂Cl₂. Fractions containing product were concentrated under reduced pressure to give 0.35 g (88%) of 28 as a white solid.

Synthesis of 2-Amino-2-(4-(2,2,3,3,4,4,5,5,6,6,7,7,8,8,8-d₁₅-octyl)phenethyl)propane-1,3-diol (112). A solution of 28 (0.35 g, 0.96 mmol, 1 equiv) in MeOH (10 mL) and 2 N aq. LiOH (2.1 mL, 4.2 equiv) was heated at reflux for 2 h. The reaction mixture was cooled to rt and the solvent removed under reduced pressure. The residue was partitioned between H₂O (10 mL) and EtOAc (100 mL). The phases were separated and the aqueous phase was extracted with EtOAc (2×, 200 mL total). The combined organics were dried over Na₂SO₄, filtered, and evaporated under reduced pressure to give a yellow solid. The solid was crystallized from EtOAc (5 mL). The crystals were collected, washed with cold EtOAc (10 mL) and dried under vacuum to give 0.11 g (36%) of 112 as a white solid, mp 117.8-118.3° C. ¹H-NMR (300 MHz, CDCl₃): δ 1.67-1.73 (m, partially obscured by H₂O, 2H), 2.54 (s, 2H), 2.59-2.64 (m, 2H), 3.51 (d, J=10.8, 2H), 3.61 (d, J=10.8, 2H), 7.10 (s, 4H). HPLC (method: Waters Atlantis T3 2.1×50 mm 3 μm C18-RP column−gradient method 5−95% ACN+0.1% formic acid in 14 min (1.0 mL/min) with 4 min hold at 95% ACN; Wavelength: 210 nm): retention time: 6.38 min; 97% purity. MS (M+H): 323.4.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention. All the patents, journal articles and other documents discussed or cited above are herein incorporated by reference.

1. A compound of the formula: 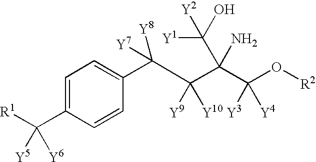
or a pharmaceutically acceptable salt thereof, wherein:
each Y is independently selected from H and D;
R¹ is —(CH₂)₆—CH₃, wherein from 1 to 15 hydrogen atoms are optionally replaced by deuterium atoms;
R² is selected from H and —P(O)(OH)₂; and
when each Y is H, R¹ contains at least one deuterium atom.

2. The compound of claim 1, wherein each of Y¹, Y², Y³ and Y⁴ is the same; each of Y⁵ and Y⁶ is the same; each of Y⁷ and Y⁸ is the same; and each of Y⁹ and Y¹⁰ is the same.

3. The compound of claim 1 or 2, wherein R¹ is selected from —(CH₂)₆—CD₃ and —(CD₂)₆—CD₃.

4. The compound of claim 1 or 2, wherein R² is —P(O)(OH)₂.

5. The compound of claim 4, wherein the compound has the (S) configuration at the carbon bearing the NH₂.

6. The compound of claim 1 or 2, wherein R² is hydrogen.

7. The compound of claim 1, selected any one of: 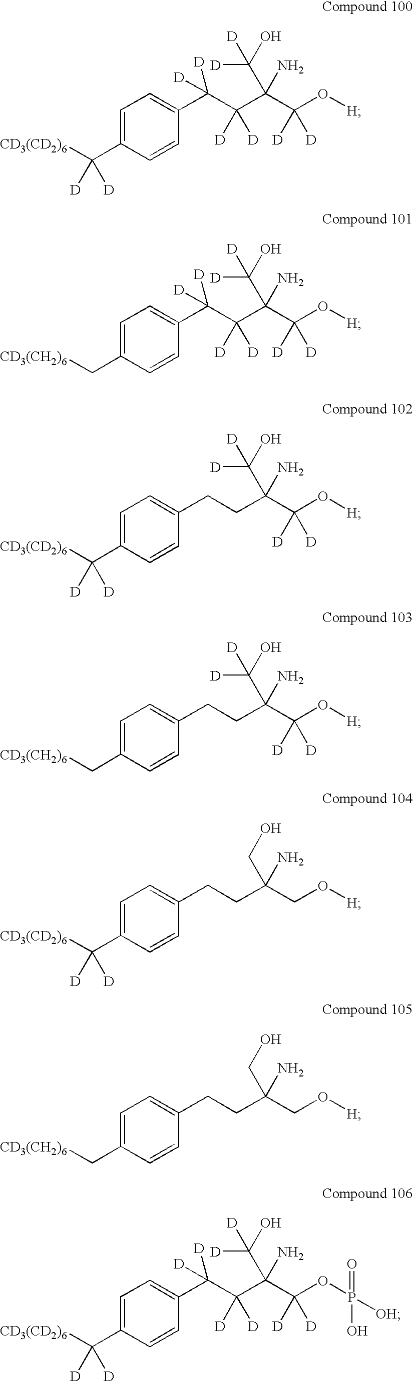 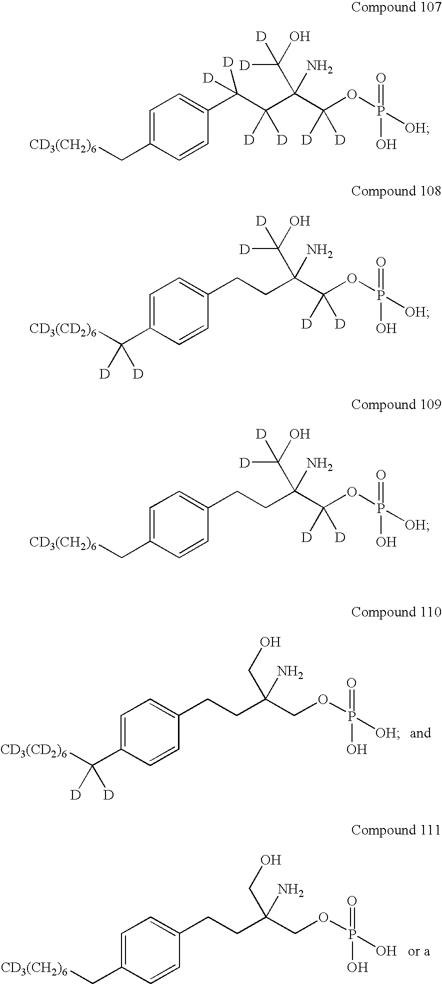
or a pharmaceutically acceptable salt of any of the foregoing.

8. The compound of claim 1, having the formula: 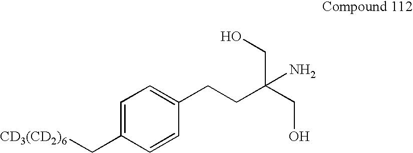
or a pharmaceutically acceptable salt thereof.

9. The compound of any one of claims 1, 2, 7 or 8, wherein any atom not designated as deuterium is present at its natural isotopic abundance.

10. A pyrogen-free pharmaceutical composition comprising a compound of the formula: 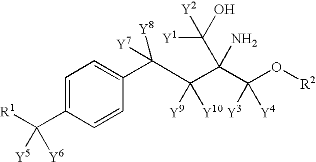
or a pharmaceutically acceptable salt thereof, wherein:
each Y is independently selected from H and D;
R¹ is —(CH₂)₆—CH₃, wherein from 1 to 15 hydrogen atoms are optionally replaced by deuterium atoms:
R² is selected from H and —P(O)(OH)₂;
when each Y is H, R¹ contains at least one deuterium atom; and
a pharmaceutically acceptable carrier.

11. The composition of claim 10 additionally comprising a second therapeutic agent selected from tacrolimus, a corticosteroid, and a cyclosporin.

12. A method of treating a disease or condition selected from multiple sclerosis (MS), inflammatory bowel disease, cancer, and ulcerative colitis, or of preventing rejection following kidney transplantation in a patient in need thereof comprising the step of administering to the patient an effective amount of a composition comprising a compound of the formula: 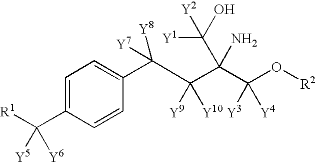
or a pharmaceutically acceptable salt thereof, wherein:
each Y is independently selected from H and D;
R¹ is —(CH₂)₆—CH₃ wherein from 1 to 15 hydrogen atoms are optionally replaced by deuterium atoms;
R² is selected from H and —P(O)(OH)₂;
when each Y is H, R¹ contains at least one deuterium atom; and
a pharmaceutically acceptable carrier.

13. The method of claim 12, wherein the method is used to preventing rejection following kidney transplantation, the method comprising the additional step of co-administering to the patient in need thereof a second therapeutic agent selected from tacrolimus, a corticosteroid, and a cyclosporin.

14. The compound of claim 3, wherein R² is —P(O)(OH)₂.

15. The compound of claim 14, wherein the compound has the (S) configuration at the carbon bearing the NH₂.

16. The compound of claim 3, wherein R² is hydrogen.

17. The compound of claim 3, wherein any atom not designated as deuterium is present at its natural isotopic abundance.

18. The compound of claim 4, wherein any atom not designated as deuterium is present at its natural isotopic abundance.

19. The compound of claim 5, wherein any atom not designated as deuterium is present at its natural isotopic abundance.

20. The compound of claim 6, wherein any atom not designated as deuterium is present at its natural isotopic abundance.
